(19) United States
(12) Patent Application Publication
(10) Pub. No.: US 2022/0127263 A1
(43) Pub. Date: Apr. 28, 2022

(54) Novel Compounds for the Treatment, Alleviation or Prevention of Disorders Associated with Tau Aggregates

(71) Applicant: AC Immune SA, 1015 Lausanne (CH)

(72) Inventors: Sreenivasachary Nampally, Ecublens (CH); Emanuele Gabellieri, 1018 Lausanne (CH)

(21) Appl. No.: 17/434,918

(22) PCT Filed: Jan. 29, 2020
(86) PCT No.: PCT/EP2020/052088

§ 371 Date: Aug. 30, 2021

(30) Foreign Application Priority Data
Mar. 1, 2019 (EP) 19160275.4

(51) Int. Cl. C07D 471/04; A61K 45/06; C07D 498/04; A61K 31/5377

(52) U.S. Cl. CPC C07D 471/04; A61K 31/5377; C07D 498/04; A61K 45/06

(57) ABSTRACT

The present invention relates to novel compounds that can be employed in the treatment, alleviation or prevention of a group of disorders and abnormalities associated with Tau (Tubulin associated unit) protein aggregates including, but not limited to, Neurofibrillary Tangles (NFTs), such as Alzheimer's disease (AD).

### FIELD OF THE INVENTION

The present invention relates to novel compounds that can be employed in the treatment, alleviation or prevention of a group of disorders and abnormalities associated with Tau (Tubulin associated unit) protein aggregates including, but not limited to, Neurofibrillary Tangles (NFTs), such as Alzheimer's disease (AD).

### BACKGROUND OF THE INVENTION

Many aging diseases are based on or associated with extracellular or intracellular deposits of amyloid or amyloid-like proteins that contribute to the pathogenesis as well as to the progression of the disease. The best characterized amyloid protein that forms extracellular aggregates is amyloid beta (Aβ). Other examples of amyloid proteins that form extracellular aggregates are prion, ATTR (transthyretin) or ADan (ADanPP). Amyloid-like proteins, that form mainly intracellular aggregates, include, but are not limited to Tau, alpha-synuclein, TAR DNA-binding protein 43 (TDP-43), and huntingtin (htt). Diseases involving Tau aggregates are generally listed as tauopathies such as AD.

Amyloid or amyloid-like deposits result from misfolding of proteins followed by aggregation to give β-sheet assemblies in which multiple peptides or proteins are held together by inter-molecular hydrogen-bonds. While amyloid or amyloid-like proteins have different primary amino acid sequences, their deposits often contain many shared molecular constituents, in particular the presence of β-sheet quaternary structures. The association between amyloid deposits and diseases remains largely unclear. A diverse range of protein aggregates, including both those associated and not associated with disease pathologies, have been found to be toxic suggesting that the common molecular features of amyloid are implicated or responsible for disease on-set (Bucciantini et al., Nature, 2002, 416, 507-11). Various multimers of β-sheet aggregated peptides or proteins have also been associated with toxicity for different peptides or proteins ranging from dimers, through to soluble low molecular weight oligomers, protofibrils or insoluble fibrillar deposits.

Alzheimer's disease (AD) is a neurological disorder primarily thought to be caused by amyloid plaques, an extracellular accumulation of abnormal deposit of (amyloid-beta) Aβ aggregates in the brain. The other major neuropathological hallmarks in AD are the intracellular neurofibrillary tangles (NFT) that originate by the aggregation of the hyperphosphorylated Tau protein, misfolded Tau or pathological Tau and its conformers. AD shares its etiopathology with many neurodegenerative tauopathies, in particular with specified types of frontotemporal dementia (FTD). The Tau protein is a freely soluble, “naturally unfolded” protein that binds avidly to microtubuli (MT) to promote their assembly and stability. MT are of major importance for the cytoskeletal integrity of neurons—and thereby for the proper formation and functioning of neuronal circuits, hence for learning and memory. The binding of Tau to MT is controlled by dynamic phosphorylation and de-phosphorylation, as demonstrated mainly in vitro and in non-neuronal cells. In AD brain, Tau pathology (tauopathy) develops later than amyloid pathology, but it is still discussed controversially if Aβ protein is the causative agent in AD which constitutes the essence of the so-called amyloid cascade hypothesis (Hardy et al., Science 1992, 256, 184-185; Musiek et al., Nature Neurosciences 2015, 18(6), 800-806). The exact mechanisms that link amyloid to Tau pathology remain largely unknown, but are proposed to involve activation of neuronal signaling pathways that act on or by GSK3 and cdk5 as the major “Tau-kinases” (Muyllaert et al., Rev. Neurol. (Paris), 2006, 162, 903-7; Muyllaert et al., Genes Brain and Behav. 2008, Suppl 1, 57-66). Even if the tauopathy develops later than amyloid, it is not just an innocent side-effect but a major pathological executer in AD. In experimental mouse models the cognitive defects caused by amyloid pathology are nearly completely alleviated by the absence of Tau protein (Roberson et al., Science, 2007, 316(5825), 750-4) and the severity of cognitive dysfunction and dementia correlates with the tauopathy, not with amyloid pathology.

Diseases involving Tau aggregates are generally listed as tauopathies and they include, but are not limited to, Alzheimer's disease (AD), familial AD, PART (primary age-related Tauopathy), Creutzfeldt-Jacob disease, dementia pugilistica, Down's Syndrome, Gerstmann-Sträussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease. (Williams et al., Intern. Med. J., 2006, 36, 652-60; Kovacs et al., J Neuropathol Exp Neurol. 2008; 67(10): 963-975; Higuchi et al., Neuropsychopharmacology—5th Generation of Progress, 2002, Section 9, Chapter 94: 1339-1354; Hilton et al., Acta Neuropathol. 1995; 90(1):101-6; Iqbal et al., Biochimica et Biophysica Acta 1739 (2005) 198—210; McQuaid et al., Neuropathol Appl Neurobiol. 1994 April; 20(2):103-10; Vossel et al., Lancet Neurol 2017; 16: 311-22; Stephan et al., Molecular Psychiatry (2012) 17, 1056-1076; Anderson et al., Brain (2008), 131, 1736-1748; Savica et al., JAMA Neurol. 2013; 70(7):859-866; Brown et al. Molecular Neurodegeneration 2014, 9:40; El Khoury et al., Front. Cell. Neurosci., 2014, Volume 8, Article22: 1-18; Tanskanen et al., Ann. Med. 2008; 40(3):232-9; Gupta et al., CAN J OPHTHALMOL—VOL. 43, NO. 1, 2008: 53-60; Dickson et al., Int J Clin Exp Pathol 2010; 3(1):1-23; Fernandez-Nogales et al., Nature Medicine, 20, 881-885 (2014); Bi et al., Nature Communications volume 8, Article number: 473 (2017); Murray et al., Biol Psychiatry. 2014 April 1; 75(7): 542-552).

Of all the agents in clinical trials for the treatment of Alzheimer's disease in 2017, the ones targeting Tau are very scarce and represent only 8% of the Phase II clinical trials (Cummings et al., Alzheimer's & Dementia: Translational Research & Clinical Interventions 3 (2017) 367-384). Current therapeutic approaches that target Tau protein comprise mainly antibody-based approaches with the main limitation of targeting only extracellular Tau. Among the approaches using small molecules, several Tau kinase inhibitors have been developed, despite being very challenging with respect to toxicity and specificity. Nevertheless, currently only one kinase inhibitor, Nilotinib, is tested in clinical trials. Lastly, among the Tau aggregation inhibitors only one, LMTX, is currently in clinical trials (Cummings et al., 2017). Although in recent years, Tau-based treatments have become a point of increasing focus, there is still a big need for additional therapeutic agents that target the pathological Tau conformers that are known or presumed to cause tauopathies.

WO2011/128455 refers to specific compounds which are suitable for treating disorders associated with amyloid proteins or amyloid-like proteins.

WO2010/080253 refers to dipyridyl-pyrrole derivative compounds which are useful in the treatment of diseases amenable to protein kinase signal transduction inhibition, regulation and/or modulation.

### SUMMARY OF THE INVENTION

It was an object of the present invention to provide compounds that can be employed in the treatment, alleviation or prevention of a group of disorders and abnormalities associated with Tau protein aggregates including, but not limited to, NFTs, such as Alzheimer's disease (AD). Furthermore, there exists a need in the art for compounds which can be used as therapeutic agents for decreasing Tau aggregates by recognizing aggregated Tau and disaggregating Tau, for example by changing the Tau aggregate molecular conformation.

Some of the compounds of formula (I) display high capability in decreasing Tau aggregates by recognizing aggregated Tau and disaggregating Tau, for example by changing the Tau aggregate molecular conformation. Due to their unique design features, these compounds display properties such as appropriate lipophilicity and molecular weight, brain uptake and pharmacokinetics, cell permeability, solubility and metabolic stability, in order to be a successful medicament for the treatment, alleviation or prevention of tauopathies.

The accumulation of Tau NFT lesions has been shown to correlate well with cognitive deficits in AD, both through histopathological analyses as well as through in vivo Tau PET imaging. The compounds of this invention disaggregate pre-existing Tau aggregates and can therefore be expected to prevent or reduce the associated cognitive deficits in AD.

Ultrastructural analyses (Masters C L, et al. Neuronal origin of a cerebral amyloid: neurofibrillary tangles of Alzheimer's disease contain the same protein as the amyloid of plaque cores and blood vessels. EMBO J. 1985; 4(11):2757-63) have shown that Tau inclusions are composed of paired helical filaments (PHF) or straight filaments (SF). High resolution structural analyses by cryo-EM have shown that these filaments are composed of a core region comprising amino acids 306-378 of Tau which adopt a cross beta/beta-helix structure (Fitzpatrick A W P, et al. Cryo-EM structures of tau filaments from Alzheimer's disease. Nature. 2017; 547(7662):185-90). The compounds of this invention can recognize aggregated Tau and disaggregate Tau, for example, by changing the Tau aggregate molecular conformation, and can therefore be expected to facilitate Tau clearance.

The present invention discloses novel compounds of formula (I) having capabilities to decrease Tau aggregates, recognize aggregated Tau and disaggregate Tau, for example, by changing the Tau aggregate molecular conformation. The present invention provides methods for the treatment of disorders and abnormalities associated with Tau protein aggregates including, but not limited to, NFTs, using a compound of formula (I) or a pharmaceutical composition thereof.

The present invention further provides a pharmaceutical composition comprising a compound of formula (I) and a pharmaceutically acceptable carrier or excipient.

Some of the compounds of formula (I) (a) display high capability in decreasing Tau aggregates by recognizing aggregated Tau and disaggregating Tau, for example by changing the Tau aggregate molecular conformation, and/or (b) prevent the formation of Tau aggregates, and/or (c) interfere intracellularly with Tau aggregates, and/or (d) reduce neuroinflammatory markers. While not wishing to be bound by theory, it is assumed that the compounds of formula (I) inhibit the Tau aggregation or disaggregate preformed Tau aggregates including when present intracellularly. Due to their unique design features, these compounds display properties such as appropriate lipophilicity and molecular weight, brain uptake and pharmacokinetics, cell permeability, solubility and metabolic stability, in order to be a successful medicament for the treatment, alleviation or prevention of tauopathies.

The present invention is summarized in the following items:
1. A compound of formula (I):

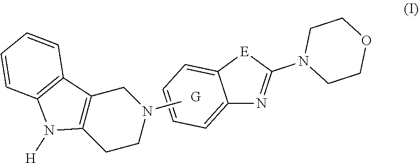

or a pharmaceutically acceptable salt, thereof;
wherein
E is selected from the group consisting of O and S; and
G is selected from the group consisting of a benzene ring, a pyrimidine ring and a pyridine ring.
2. The compound according to item 1, which is a compound of formula (Ia):

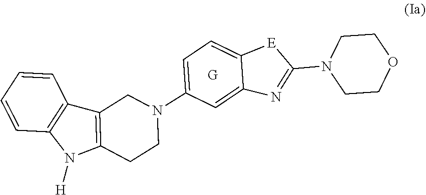

wherein E, and G are as defined in item 1.
3. The compound according to item 1 or 2, wherein G is a benzene ring.
4. The compound according to any one of items 1 to 3, wherein E is O.
5. A pharmaceutical composition comprising a compound as defined in any one of items 1 to 4 and optionally a pharmaceutically acceptable carrier or excipient.
6. The compound as defined in any one of items 1 to 4 for use as a medicament.
7. The compound as defined in any one of items 1 to 4 for use in the treatment, alleviation or prevention of a disorder or abnormality associated with Tau protein aggregates.
8. The compound as defined in any one of items 1 to 4 for use in decreasing tau aggregation.
9. A method of treating, preventing or alleviating a disorder or abnormality associated with Tau protein aggregates, the method comprising administering an effective amount of a compound as defined in any one of items 1 to 4 to a subject in need thereof.
10. A method of decreasing Tau aggregation, the method comprising administering an effective amount of a compound as defined in any one of items 1 to 4 to a subject in need thereof.
11. The use of a compound as defined in any one of items 1 to 4 in the manufacture of a medicament for treating, preventing or alleviating a disorder or abnormality associated with Tau protein aggregates.
12. The use of a compound as defined in any one of items 1 to 4 in the manufacture of a medicament for decreasing Tau aggregation.
13. A mixture comprising a compound as defined in any one of items 1 to 4 and at least one further biologically active compound selected from a therapeutic agent different from the compound as defined in any one of items 1 to 4, wherein the mixture further comprises at least one of a pharmaceutically acceptable carrier, a diluent and an excipient.
14. The mixture according to item 15, wherein the further biologically active compound is a compound used in the treatment of amyloidosis.
15. The mixture according to item 13 or 14, wherein the compound and/or the further biologically active compound is/are present in a therapeutically effective amount.
16. The mixture according to any one of items 13 to 15, wherein the further biologically active compound is selected from the group consisting of compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), α-secretase activators, β- and γ-secretase inhibitors, glycogen synthase kinase 3 inhibitors, O-glucanase (OGA) inhibitors, neurotransmitter, β-sheet breakers, attractants for amyloid beta clearing/depleting cellular components, inhibitors of N-terminal truncated amyloid beta including pyroglutamated amyloid beta 3-42, anti-inflammatory molecules, or cholinesterase inhibitors (ChEIs) such as tacrine, rivastigmine, donepezil, and/or galantamine, M1 agonists, other drugs including any amyloid or Tau modifying drug and nutritive supplements, an antibody, including any functionally equivalent antibody or functional parts thereof or a vaccine.
17. The mixture according to item 16, wherein the further biologically active compound is a cholinesterase inhibitor (ChEI).
18. The mixture according to item 16, wherein the further biologically active compound is selected from the group consisting of tacrine, rivastigmine, donepezil, galantamine, niacin and memantine.
19. The mixture according to item 16, wherein the further biologically active compound is an antibody, particularly a monoclonal antibody, including any functionally equivalent antibody or functional parts thereof.
20. The mixture according to any one of items 13 to 19, wherein the compound and/or the further biologically active compound is/are present in a therapeutically effective amount.
21. The compound for use according to item 7, the method according to item 9, or the use according to item 11, wherein the disorder is selected from Alzheimer's disease (AD), familial AD, Primary Age-Related Tauopathy (PART), Creutzfeldt-Jacob disease, dementia pugilistica, Down's Syndrome, Gerstmann-Sträussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease, preferably Alzheimer's disease (AD), corticobasal degeneration (CBD), Pick's disease (PiD), and progressive supranuclear palsy (PSP).
22. Use of the compound as defined in any one of items 1 to 4 as an analytical reference or an in vitro screening tool.
23. The compound as defined on any one of items 1 to 4 for use in preventing the formation of Tau aggregates and/or for use in inhibiting Tau aggregation.
24. The compound as defined on any one of items 1 to 4 for use in interfering intracellulary with Tau aggregates.
25. A method of decreasing Tau aggregation, the method comprising administering an effective amount of a compound as defined in any one of items 1 to 4 to a subject in need thereof.
26. A method of preventing the formation of Tau aggregates and/or of inhibiting Tau aggregation, the method comprising administering an effective amount of a compound as defined in any one of items 1 to 4 to a subject in need thereof.
27. A method of interfering intracellulary with Tau aggregates and/or of inhibiting Tau aggregation, the method comprising administering an effective amount of a compound as defined in any one of items 1 to 4 to a subject in need thereof.
28. A method of reducing neuroinflammatory markers, the method comprising administering an effective amount of a compound as defined in any one of items 1 to 4 to a subject in need thereof.
29. The compound as defined on any one of items 1 to 4 for use in reducing neuroinflammatory markers.

### Definitions

Within the meaning of the present application the following definitions apply: “Hal” or “halogen” refers to F, Cl, Br, and I.

The term “polymorphs” refers to the various crystalline structures of the compounds of the present invention. This may include, but is not limited to, crystal morphologies (and amorphous materials) and all crystal lattice forms. Salts of the present invention can be crystalline and may exist as more than one polymorph.

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid salts thereof. Examples of pharmaceutically acceptable salts include, but are not limited to, mineral or organic acid salts of the amine residue and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric acid and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. The pharmaceutically acceptable salts of the present invention can be synthesized from the parent compound by conventional chemical methods. Generally, such salts can be prepared by reacting the compounds with a stoichiometric amount of the appropriate acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18th ed., Mack Publishing Company, Easton, Pa., 1990, p. 1445, the disclosure of which is hereby incorporated by reference.

The compounds of the present invention can also be provided in the form of a prodrug, namely a compound which is metabolized in vivo to the active metabolite. As used hereinafter in the description of the invention and in the claims, the term “prodrug” means any covalently bonded compound which releases the active parent pharmaceutical due to in vivo biotransformation. The reference by Goodman and Gilman (The Pharmacological Basis of Therapeutics, 8 ed, McGraw-Hill, Int. Ed. 1992, “Biotransformation of Drugs”, p 13-15) describing prodrugs generally is hereby incorporated herein by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The patients or subjects in the present invention are typically animals, particularly mammals, more particularly humans.

“Tau” as used herein refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitination, sumoylation and oxidation.

“Aggregated Tau” refers to aggregated monomers of Tau peptides or proteins which are folded into the oligomeric or polymeric structures.

“Neurofibrillary Tangles” (NFTs) as used herein refer to insoluble aggregates of the hyperphosphorylated Tau protein containing paired helical filaments (PHF) and straight filaments. Their presence is a hallmark of AD and other diseases known as tauopathies.

The terms “antibody” or “antibodies” as used herein is an art recognized term and is understood to refer to molecules or active fragments of molecules that bind to known antigens, or refer particularly to immunoglobulin molecules and to antigen binding portions of immunoglobulin molecules. In particular the mixture of the present invention includes the compounds of the present invention and anti Tau or anti Abeta antibodies.

The term “functional equivalent antibody or functional part thereof” as used herein is understood to refer to an equivalent molecule or active fragments of a molecule that binds to a known antigen, or refer particularly to an immunoglobulin molecule and to antigen binding portions of an immunoglobulin molecule and has essentially the same (biological) activity as the antibody from which it is derived.

The “vaccine” or “vaccines” reported in the mixtures of the present invention, are in particular Tau or Abeta vaccines.

The definitions and preferred definitions given in the “Definition”-section apply to all of the embodiments described below unless stated otherwise.

### DETAILED DESCRIPTION OF THE INVENTION

The compounds of the present invention will be described in the following. It is to be understood that all possible combinations of the following definitions are also envisaged.

In one embodiment, the present invention relates to a compound of formula (I):

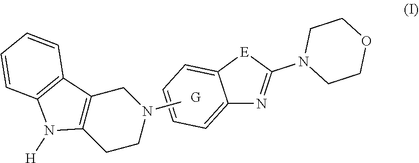

and all pharmaceutically acceptable salts, prodrugs, hydrates, solvates and polymorphs thereof.

A preferred embodiment of the compound of formula (I) is

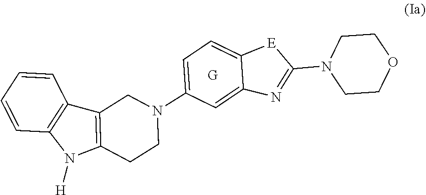

The following definitions apply to the formula (I) and their preferred embodiments, as appropriate.

E is selected from the group consisting of O and S, more preferably E is O.

Therefore

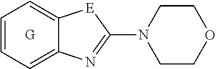

covers the following preferred embodiments

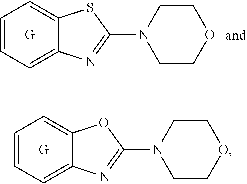 [SMILES: c1ccc2sc(N3CCOCC3)nc2c1 ; "and" ; c1ccc2oc(N3CCOCC3)nc2c1]

more preferably

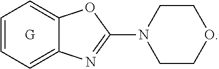 [SMILES: c1ccc2oc(N3CCOCC3)nc2c1]

G is selected from the group consisting of a benzene ring, a pyrimidine ring and a pyridine ring. More preferably G is benzene.

Therefore

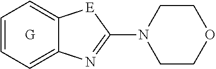

covers the following preferred embodiments

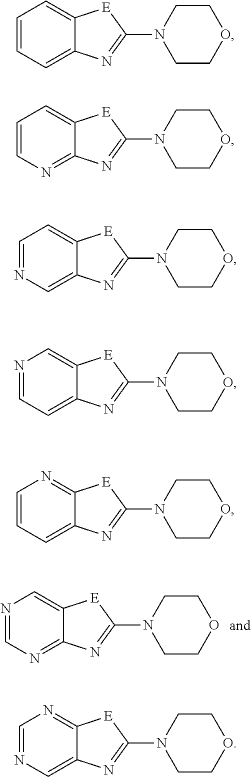

More preferably

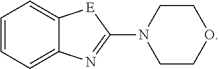

In a further preferred embodiment,

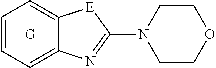

is selected from

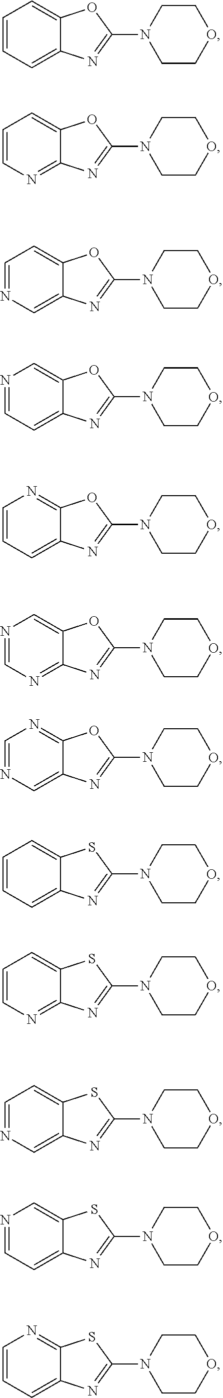

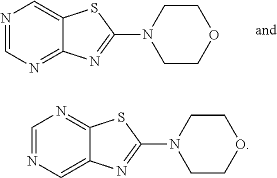 [SMILES: c1ncc2sc(N3CCOCC3)nc2n1 ; c1ncc2nc(N3CCOCC3)sc2n1]

More preferably

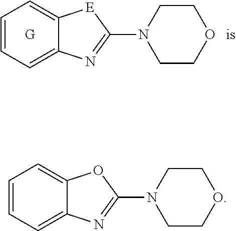

Preferred compounds are also illustrated in the examples.

Any combination of the embodiments, preferred embodiments and more preferred embodiments disclosed herein is also envisaged in the present invention.

Pharmaceutical Compositions

While it is possible for the compounds of the present invention to be administered alone, it is preferable to formulate them into a pharmaceutical composition in accordance with standard pharmaceutical practice. Thus, the invention also provides a pharmaceutical composition which comprises a therapeutically effective amount of a compound of formula (I) optionally in admixture with a pharmaceutically acceptable carrier, diluent, adjuvant or excipient.

Pharmaceutically acceptable excipients are well known in the pharmaceutical art, and are described, for example, in Remington's Pharmaceutical Sciences, 15th Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients that may be used in the formulation of the pharmaceutical composition of the present invention may comprise, for example, carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-ß-cyclodextrin, polyvinylpyrrolidone, low melting waxes, and ion exchange resins.

The routes for administration (delivery) of the compounds of the invention include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compounds of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compound and a suitable powder base such as lactose or starch.

Alternatively, the compounds of the present invention can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH was adjusted, sterile saline, or, preferably, as solutions in isotonic, pH was adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

A proposed dose of the compounds according to the present invention for administration to a human (of approximately 70 kg body weight) is 0.1 mg to 3 g, 0.1 mg to 2 g, 0.1 mg to 1 g, preferably 1 mg to 500 mg of the active ingredient per unit dose. The unit dose may be administered, for example, 1 to 4 times per day. The dose will depend on the route of administration. It will be appreciated that it may be necessary to make routine variations to the dosage depending on the age and weight of the patient as well as the severity of the condition to be treated. The precise dose and route of administration will ultimately be at the discretion of the attendant physician or veterinarian.

The compounds of the invention may also be used in combination with other therapeutic agents. When a compound of the invention is used in combination with a second therapeutic agent active against the same disease, the dose of each compound may differ from that when the compound is used alone.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

The pharmaceutical compositions of the invention can be produced in a manner known per se to the skilled person as described, for example, in Remington's Pharmaceutical Sciences, 15th Ed., Mack Publishing Co., New Jersey (1975).

The diseases or conditions that can be treated, alleviated or prevented with the compounds of the present invention are disorders or abnormalities associated with Tau protein aggregates such as neurodegenerative disorders. Examples of diseases and conditions which can be treated, alleviated or prevented are caused by or associated with the formation of neurofibrillary lesions. This is the predominant brain pathology in tauopathy. The diseases and conditions comprise a heterogeneous group of neurodegenerative diseases or conditions including diseases or conditions which show co-existence of Tau and amyloid pathologies.

Examples of the diseases and conditions which can be treated, alleviated or prevented include, but are not limited, to Alzheimer's disease (AD), familial AD, PART (Primary Age-Related Tauopathy), Creutzfeldt-Jacob disease, dementia pugilistica, Down's Syndrome, Gerstmann-Sträussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease. Preferably the diseases and conditions which can be treated, alleviated or prevented include Alzheimer's disease (AD), as well as other neurodegenerative tauopathies such as Creutzfeldt-Jacob disease, dementia pugilistica, amyotrophic lateral sclerosis (ALS), argyrophilic grain disease, corticobasal degeneration (CBD), frontotemporal dementia with Parkinsonism linked to chromosome 17 (FTDP-17), Pick's disease (PiD), progressive supranuclear palsy (PSP), tangle predominant dementia, Parkinson dementia complex of Guam, Hallervorden-Spatz disease, chronic traumatic encephalopathy (CTE), traumatic brain injury (TBI), and other frontotemporal lobar degeneration. More preferably Alzheimer's disease (AD), corticobasal degeneration (CBD), Pick's disease (PiD), and progressive supranuclear palsy (PSP).

The compounds of the present invention can also be employed to decrease protein aggregation, in particular Tau aggregation. The ability of a compound to decrease of Tau aggregation can, for example, be determined using the ThT assay (Hudson et al., FEBS J., 2009, 5960-72).

The compounds of the present invention can be used as an analytical reference or an in vitro screening tool for characterization of tissue with Tau pathology and for testing of compounds targeting Tau pathology on such tissue.

The compounds according to the present invention can also be provided in the form of a mixture with at least one further biologically active compound and/or a pharmaceutically acceptable carrier and/or a diluent and/or an excipient. The compound and/or the further biologically active compound are preferably present in a therapeutically effective amount.

The nature of the further biologically active compound will depend on the intended use of the mixture. The further biologically active substance or compound may exert its biological effect by the same or a similar mechanism as the compound according to the invention or by an unrelated mechanism of action or by a multiplicity of related and/or unrelated mechanisms of action.

Generally, the further biologically active compound may include neutron-transmission enhancers, psychotherapeutic drugs, acetylcholineesterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists. In particular, the further biologically active compound can be selected from the group consisting of a compound used in the treatment of amyloidosis, compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), α-secretase activators, β- and γ-secretase inhibitors, glycogen synthase kinase 3 inhibitors, O-glucanase (OGA) inhibitors, neurotransmitter, β-sheet breakers, attractants for amyloid beta clearing/depleting cellular components, inhibitors of N-terminal truncated amyloid beta including pyroglutamated amyloid beta 3-42, anti-inflammatory molecules, or cholinesterase inhibitors (ChEIs) such as tacrine, rivastigmine, donepezil, and/or galantamine, M1 agonists, other drugs including any amyloid or Tau modifying drug and nutritive supplements, an antibody, including any functionally equivalent antibody or functional parts thereof, or a vaccine.

In a further embodiment, the mixtures according to the invention may comprise niacin or memantine together with a compound according to the present invention and, optionally, a pharmaceutically acceptable carrier and/or a diluent and/or an excipient.

In still another embodiment of the invention mixtures are provided that comprise as a further biologically active compound “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with a compound according to the invention and, optionally, a pharmaceutically acceptable carrier and/or a diluent and/or an excipient.

Other compounds that can be suitably used in mixtures in combination with the compound according to the present invention are, for example, described in WO 2004/058258 (see especially pages 16 and 17) including therapeutic drug targets (pages 36 to 39), alkanesulfonic acids and alkanolsulfuric acids (pages 39 to 51), cholinesterase inhibitors (pages 51 to 56), NMDA receptor antagonists (pages 56 to 58), estrogens (pages 58 to 59), non-steroidal anti-inflammatory drugs (pages 60 and 61), antioxidants (pages 61 and 62), peroxisome proliferators-activated receptor (PPAR) agonists (pages 63 to 67), cholesterol-lowering agents (pages 68 to 75), amyloid inhibitors (pages 75 to 77), amyloid formation inhibitors (pages 77 to 78), metal chelators (pages 78 and 79), anti-psychotics and anti-depressants (pages 80 to 82), nutritional supplements (pages 83 to 89) and compounds increasing the availability of biologically active substances in the brain (see pages 89 to 93) and prodrugs (pages 93 and 94), which document is incorporated herein by reference.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., 14C, isotopes are particularly preferred for their ease of preparation and delectability. ¹⁸F-labeled compounds are particularly suitable for imaging applications such as PET. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

The compounds of the present invention can be synthesized by one of the general methods shown in the following schemes. These methods are only given for illustrative purposes and should not to be construed as limiting.

### General Synthetic Schemes for the Preparation of Building Blocks of this Invention

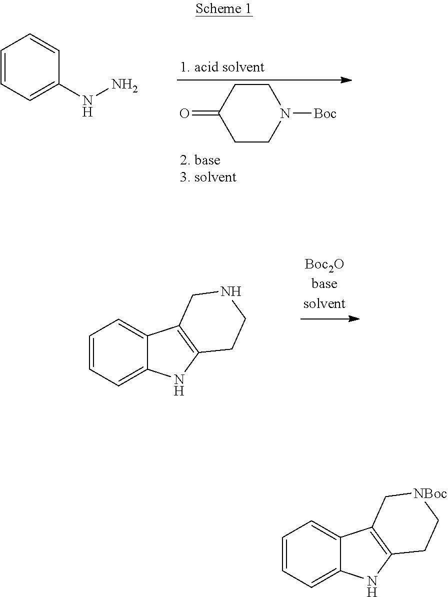 [SMILES: NNc1ccccc1 ; CC(C)(C)OC(=O)N1CCC(=O)CC1 ; c1ccc2c3c([nH]c2c1)CCNC3 ; CC(C)(C)OC(=O)N1CCc2[nH]c3ccccc3c2C1]

Heating of commercially available phenylhydrazine derivatives with commercially available tert.-butyl 4-oxopiperidine-1-carboxylate in a suitable solvent under acidic conditions (Fischer-Indole synthesis) afforded the tricyclic 2,3,4,5-tetrahydro-1H-pyrido[4,3-b]indole building blocks after purification. The tricyclic building block was further treated with Boc₂O to selectively protect the aliphatic, secondary amine moiety and was obtained after purification.

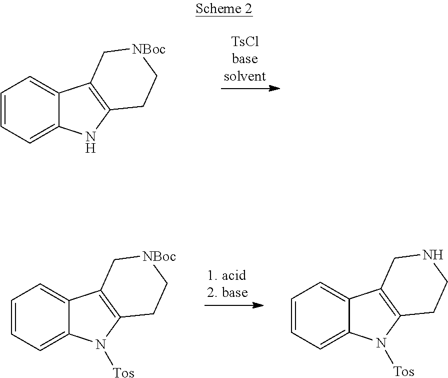

The NH-moiety of the indole moiety was then treated with tosyl chloride in an appropriate solvent using a suitable base to afford N-tosyl derivatives after purification. The Boc-protecting group was removed by acid treatment in an appropriate solvent to afford the desired tricyclic 2,3,4,5-tetrahydro-1H-pyrido[4,3-b]indole building block after purification. In case there was no base treatment, the corresponding salts were obtained.

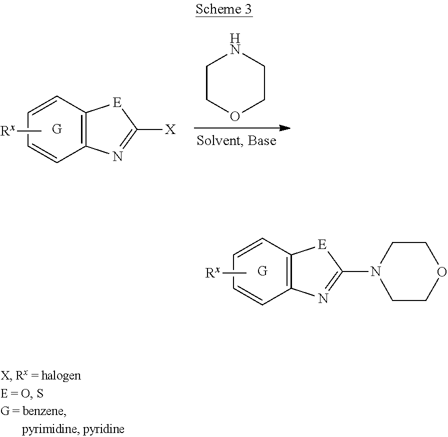

Suitable benzothiazole (G=Ph), benzoxazole (G=Ph), thiazolopyridine (G=Py), oxazolopyridine (G=Py), thiazolopyrimidine (G=pyrimidine) or oxazolopyrimidine (G=pyrimidine) derivatives containing two halogen (Br, CI) atoms, were treated with morpholine in an appropriate solvent and with an additional base. The leaving group X was replaced via nucleophilic substitution by the secondary amines to afford the corresponding amino-substituted derivatives after purification.

### General Synthetic Scheme for the Preparation of Compounds of this Invention

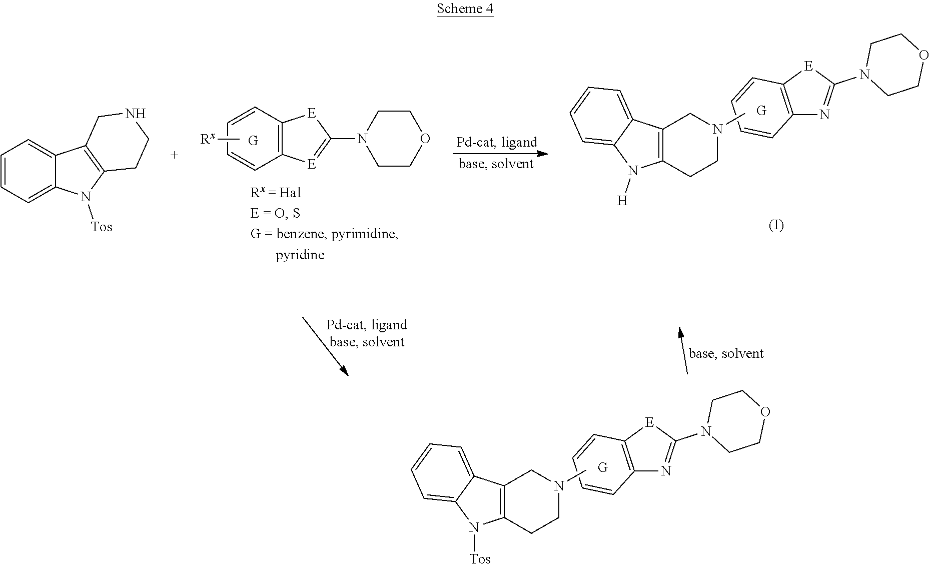

The tricyclic building block containing a N-tosyl group at the indole moiety was coupled with amino substituted benzothiazole (G=Ph), benzoxazole (G=Ph), thiazolopyridine (G=Py), oxazolopyridine (G=Py), thiazolopyrimidine (G=pyrimidine) or oxazolopyrimidine (G=pyrimidine) derivatives via palladium chemistry with a suitable palladium catalyst (tris(dibenzylideneacetone)dipalladium(0); Pd₂(dba)₃), ligand (2-dicyclohexylphosphino-2′,6′-diisopropoxybiphenyl; RuPhos) and base (sodium tert.-butoxide; NaOtBu) in a suitable solvent (1,4-dioxane) to afford the desired compounds of formula (I) after purification. Alternatively, the tricyclic building block containing a N-tosyl group at the indole moiety was coupled with amino substituted benzothiazole (G=Ph), benzooxazole (G=Ph), thiazolopyridine (G=Py), oxazolopyridine (G=Py), thiazolopyrimidine (G=pyrimidine), oxazolopyrimidine (G=pyrimidine), derivatives via palladium chemistry with a suitable palladium catalyst (tris(dibenzylideneacetone)dipalladium(0); Pd₂(dba)₃), ligand (2-dicyclohexylphosphino-2′,6′-diisopropoxybiphenyl; RuPhos) and a weaker base (caesium carbonate; Cs₂CO₃) in a suitable solvent (1,4-dioxane) to afford the N-tosyl protected compounds after purification. The tosyl-protecting group was then removed using a suitable base (caesium carbonate; Cs₂CO₃) in a suitable solvent system (2-methyl THF, methanol) at elevated temperature (reflux) to afford the desired compounds of formula (I) after purification.

The disaggregation of Tau K18 may be measured using any suitable assay known in the art. A standard in vitro assay for measuring the disaggregation capacity is described.

### EXAMPLES

All reagents and solvents were obtained from commercial sources and used without further purification. ¹H NMR spectra were recorded on Bruker AV-300 and 400 MHz spectrometer in deuterated solvents. Chemical shifts (δ) are reported in parts per million and coupling constants (J values) in hertz. Spin multiplicities are indicated by the following symbols: s (singlet), d (doublet), t (triplet), q (quartet), m (multiplet), bs (broad singlet). Mass spectra were obtained on an Agilent 1290 Infinity II spectrometer with a 6130 Chemstation and an Agilent 1200 Infinity II spectrometer with a 6130 Chemstation. GC-MS data were collected using an Agilent 7890B gas chromatograph and 5977B mass spectrometer. Infrared spectra were obtained on a PerkinElmer spectrometer. Chromatography was performed using silica gel (Fluka: Silica gel 60, 0.063-0.2 mm) and suitable solvents as indicated in specific examples. Flash purification was conducted with a Biotage Isolera with HP-Sil or KP-NH SNAP cartridges (Biotage) and the solvent gradient indicated in specific examples. Thin layer chromatography (TLC) was carried out on silica gel plates with UV detection.

### Preparative Example 1

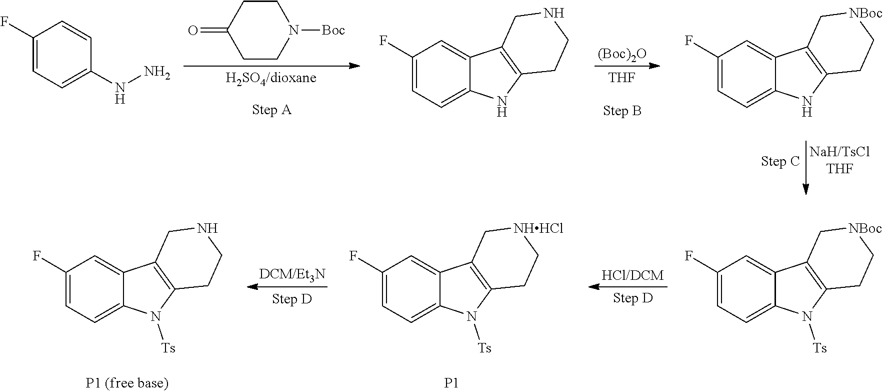

Step A

To a solution of 4-fluorophenyl hydrazine (1 g, 7.9 mmol) and tert-butyl 4-oxopiperidine-1-carboxylate (1.2 g, 8.3 mmol) in 1,4-dioxane (10 mL) was added conc. H₂SO₄ (1 mL) at ice bath temperature. Then the reaction mixture was heated at 110° C. for 3 h. The reaction mixture was cooled to room temperature, the precipitate was filtered off. The solid was dissolved in water basified with NaOH solution and extracted with DCM (dichloromethane). The organic phase was separated and dried over Na₂SO₄ and the solvent was removed to give the title compound as a pale yellow solid (950 mg, 59%).

MS: 191 (M+H)⁺.

¹H-NMR (400 MHz, DMSO-d₆) δ=10.91 (s, 1H), 7.23-7.24 (m, 1H), 7.09-7.09 (m, 1H), 6.80-6.81 (m, 1H), 3.91 (s, 2H), 3.11 (t, 2H), 2.75 (d, 2H).

Step B

To a solution of the title compound from Step A above (0.95 g, 4.77 mmol) in THF (tetrahydrofuran) was added di-tert-butyl dicarbonate (Boc₂O) (1.5 g) and the mixture was stirred overnight. After the completion of the reaction as evidenced by TLC, the solvent was removed and the crude reaction mixture was purified on a silica gel column using a Biotage Isolera One purification system employing an EtOAc/heptane gradient (10/80=>80/20) to afford the title compound as a pale yellow gummy liquid (1.1 g, 78%).

MS: 291 (M+H)⁺.

¹H-NMR (400 MHz, DMSO-d₆) δ=11.00 (s, 1H), 7.26 (q, 1H), 7.18 (t, 1H), 6.83-6.83 (m, 1H), 4.49 (s, 2H), 3.69 (t, 2H), 2.76 (s, 2H), 1.43 (s, 9H).

Step C

To a solution of the title compound from Step B above (0.41 g, 1.41 mmol) in THF (5 mL) was added sodium hydride (0.15 g, 6.25 mmol), followed by p-toluenesulfonyl chloride (TsCl) (0.29 g, 1.45 mmol). The reaction mixture was stirred for 10 min. The mixture was dissolved in EtOAc (20 ml) and washed with water and brine and dried over Na₂SO₄. The crude product was purified on a silica gel column using a Biotage Isolera One purification system employing an EtOAc/heptane gradient (20/80=>80/20) to afford the title compound (0.45 g, 72%).

MS: 445 (M+H)⁺.

¹H-NMR (400 MHz, DMSO-d₆) δ=8.03-8.04 (m, 1H), 7.77 (d, 2H), 7.36-7.38 (m, 3H), 7.15-7.16 (m, 1H), 4.43 (s, 2H), 3.69 (t, 2H), 3.08 (s, 2H), 2.32 (s, 3H), 1.43 (s, 9H).

Step D

To a solution of the title compound from Step C above (0.42 g, 0.915 mmol) in dichloromethane was added 2N HCl (5 mL) in 1,4-dioxane. The reaction mixture was stirred overnight. After the completion of the reaction, the reaction mixture was evaporated to remove the solvent and washed with diethyl ether to afford the title compound as an off white solid (0.2 g, 58%).

MS: 345 (M+H)⁺.

¹H-NMR (400 MHz, DMSO-d₆) δ=9.61 (s, 1H), 8.01-8.02 (m, 1H), 7.82 (d, 2H), 7.45-7.45 (m, 1H), 7.39 (d, 2H), 7.20-7.21 (m, 1H), 4.25 (s, 2H), 3.49 (s, 2H), 3.35 (d, 2H), 2.34 (s, 3H).

Step E

To a solution of the title compound from Step D above (5.0 g, 13 mmol) in Dichloromethane (50 mL), was added triethylamine (5 mL) and stirred for 10 min. The reaction mixture was diluted with Dichloromethane (20 mL), washed with water (2×30 mL) and a saturated solution of NaCl (30 mL). The combined organic layer was dried over sodium sulfate and concentrated under vacuum to afford the title compound as free base (quantitative yield).

### Preparative Example 2

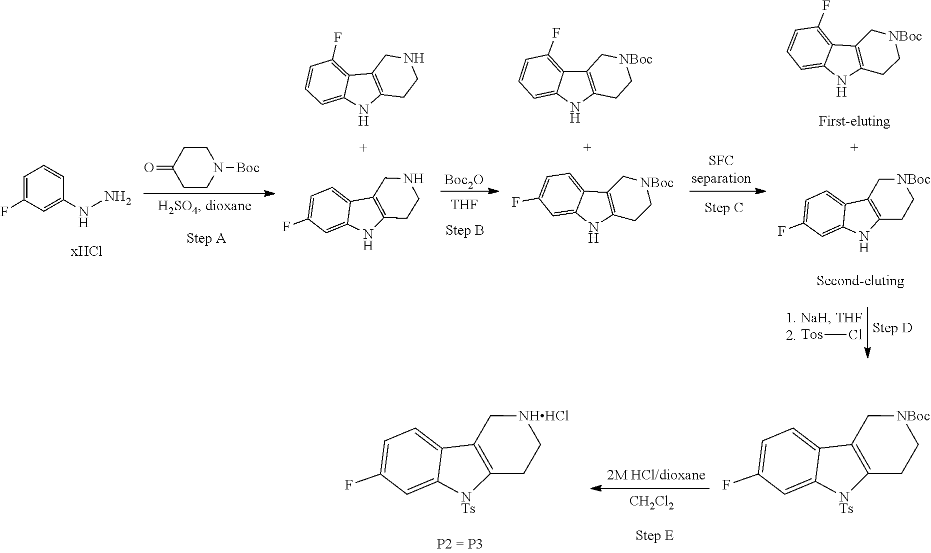

Step A

To a solution of 3-(fluorophenyl) hydrazine (1 g, 6.1 mmol) and tert-butyl 4-oxopiperidine-1-carboxylate (1.2 g, 6.1 mmol) in 1,4-dioxane (10 mL) was added conc. H₂SO₄ (1 mL) at 0° C. Then the reaction mixture was warmed to 25° C. and heated at 110° C. for 3 h. The reaction mixture was cooled to room temperature and the precipitate was filtered off. The solid was dissolved in water, basified with NaOH solution and extracted with dichloromethane. The organic phase was separated and dried over Na₂SO₄ and the solvent was removed to afford the mixture of regioisomers as a pale yellow solid (0.65 g, 56%).

MS: 191.1 (M+H)⁺.

¹H-NMR (400 MHz, DMSO-d₆) δ=10.87 (bs, 1H), 7.26-7.30 (m, 1H), 7.02-7.05 (m, 1H), 6.74-6.79 (m, 1H), 3.83 (bs, 2H), 2.99-3.02 (m, 2H), 2.65-2.66 (m, 2H).

Step B

To a solution of the mixture of regioisomers (0.65 g, 3.15 mmol) in THF was added di-tert-butyl dicarbonate (0.757 g, 3.47 mmol) and the mixture was stirred for 12 h. After the completion of the reaction (monitored by TLC), the solvent was concentrated under reduced pressure to yield the crude product. It was purified by silica gel (60-120 mesh) column chromatography using hexane: EtOAc (70:30) to afford the mixture of the regioisomers tert-butyl 7-fluoro-1,3,4,5-tetrahydro-2H-pyrido[4,3-b]indole-2-carboxylate and tert-butyl 9-fluoro-1,3,4,5-tetrahydro-2H-pyrido[4,3-b]indole-2-carboxylate as a yellow solid (0.750 g, 61%) in a ratio of ˜70:30, respectively.

MS: 291.2 (M+H)⁺.

¹H-NMR (400 MHz, DMSO-d₆) δ=11.01 (bs, 1H), 7.36-7.39 (m, 1H), 7.06-7.09 (m, 1H), 6.79-6.84 (m, 1H), 4.51 (bs, 2H), 3.68-3.71 (m, 2H), 2.74-2.76 (m, 2H), 1.38 (s, 9H).

Step C

The mixture of regioisomers (0.750 mg, 70:30) was separated by a SFC chiral column (Chiracel OJ-H; Column: X-bridge C8 (50×4.6) mm, 3.5 μm, mobile Phase A: 0.1% TFA in water, mobile phase B: 0.1% TFA acetonitrile) to afford the second-eluting title compound tert-butyl 7-fluoro-1,3,4,5-tetrahydro-2H-pyrido[4,3-b]indole-2-carboxylate as a pale yellow solid with 100% chiral purity (0.4 mg, 53%). The first-eluting title compound tert-butyl 9-fluoro-1,3,4,5-tetrahydro-2H-pyrido[4,3-b]indole-2-carboxylate was isolated as a pale yellow solid with 100% chiral purity (0.25 g, 33%).

Second-Eluting Title Compound:

MS: 291.2 (M+H)⁺.

¹H-NMR (400 MHz, DMSO-d₆) δ=11.01 (bs, 1H), 7.36-7.39 (m, 1H), 7.06-7.09 (m, 1H), 6.79-6.84 (m, 1H), 4.51 (bs, 2H), 3.68-3.71 (m, 2H), 2.74-2.77 (m, 2H), 1.44 (s, 9H).

RT=2.08 min.

First-Eluting Title Compound:

MS: 291.2 (M+H)⁺.

¹H-NMR (400 MHz, DMSO-d₆) δ=11.22 (s, 1H), 7.13 (d, 1H), 6.96-6.97 (m, 1H), 6.69-6.71 (m, 1H), 4.63 (s, 2H), 3.69-3.70 (m, 2H), 2.68-2.76 (m, 2H), 1.44 (s, 9H).

RT=1.74 min.

Step D

To a solution of the second-eluting title compound from Step C above (0.4 g, 1.37 mmol) in THF (5 mL) was added sodium hydride (0.099 mg, 4.137 mmol), followed by p-toluenesulfonyl chloride (0.288 g, 1.51 mmol). The reaction mixture was stirred for 30 minutes. The mixture was dissolved in EtOAc (20 ml) and washed with water and brine and dried over Na₂SO₄. The crude product was purified on a silica gel column using a Biotage Isolera One purification system employing an EtOAc/heptane gradient (20/80=>80/20) to afford the title compound (0.3 g, 49%).

MS: 445 (M+H)⁺.

¹H-NMR (400 MHz, chloroform-d) δ=7.92-7.94 (m, 1H), 7.68-7.70 (m, 1H), 7.25-7.29 (m, 4H), 7.00-7.04 (m, 1H), 4.50 (bs, 2H), 3.76 (bs, 2H), 3.12 (bs, 2H), 2.38 (s, 3H), 1.51 (s, 9H).

Step E

To a solution of the title compound from Step D above (0.3 g, 0.676 mmol) in dichloromethane was added 2N HCl (5 mL) in 1,4-dioxane. The reaction mixture was stirred for 12 h. After the completion of the reaction, the reaction mixture was evaporated to remove the solvent and washed with diethyl ether to afford the title compound as an off white solid (0.2 g 78%).

MS: 345 (M+H)⁺.

¹H-NMR (400 MHz, DMSO-d₆) δ=9.50 (bs, 2H), 7.80-7.87 (m, 2H), 7.78 (d, 1H), 7.57-7.61 (m, 1H), 7.40 (d, 2H), 7.18-7.23 (m, 1H), 4.27 (bs, 2H), 3.56 (bs, 2H), 3.47 (bs, 2H), 2.34 (s, 3H).

### Preparative Example 3

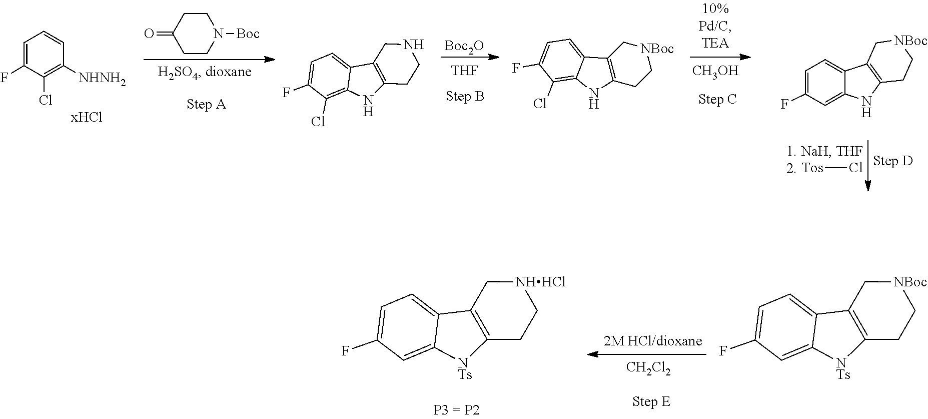

Step A

To a solution of (2-chloro-3-fluorophenyl)hydrazine (10 g, 62.5 mmol) and tert-butyl 4-oxopiperidine-1-carboxylate (12 g, 62.5 mmol) in 1,4-dioxane (100 mL) was added conc. H₂SO₄ (10 mL) at 0° C. Then the reaction mixture was warmed to 25° C. and heated at 110° C. for 3 h. The reaction mixture was cooled to room temperature and the precipitate was filtered off. The solid was dissolved in water, basified with NaOH solution and extracted with dichloromethane. The organic phase was separated and dried over Na₂SO₄ and the solvent was removed to give the title compound as a pale yellow solid (10 g, 72%).

MS: 225 (M+H)⁺.

¹H-NMR (400 MHz, DMSO-d₆) δ=11.23 (bs, 1H), 7.27-7.28 (m, 1H), 6.94-6.96 (m, 1H), 3.82 (s, 2H), 2.98-3.00 (m, 2H), 2.68 (d, 2H).

Step B

To a solution of the title compound from Step A above (10 g, 44.5 mmol) in THF (100 mL) was added di-tert-butyl dicarbonate (10.5 g, 46.5 mmol) and the mixture was stirred for 12 h. After the completion of the reaction (monitored by TLC), the solvent was concentrated under reduced pressure to yield the crude product. It was purified by silica gel (60-120 mesh) column chromatography to afford the title compound as a yellow solid (12 g, 85%).

MS: 325.1 (M+H)⁺.

¹H-NMR (400 MHz, DMSO-d₆) δ=11.43 (s, 1H), 7.36-7.38 (m, 1H), 6.97-7.00 (m, 1H), 4.51 (s, 2H), 3.68-3.69 (m, 2H), 2.76-2.78 (m, 2H), 1.43 (s, 9H).

Step C

To a solution of the title compound from Step B above (5 g, 15.3 mmol) in dry methanol (50 mL) was added triethylamine (6.74 mL, 46.18 mmol) and 10% Pd/C (0.2 mg, 20% wt). Hydrogenation was conducted under 10 bar pressure for 16 hours. The reaction mixture was filtered through a celite pad and concentrated under vacuum to afford the title compound (4 g, 90%).

MS: 291.2 (M+H)⁺.

¹H-NMR (400 MHz, DMSO-d₆) δ=11.01 (bs, 1H), 7.36-7.39 (m, 1H), 7.06-7.09 (m, 1H), 6.79-6.84 (m, 1H), 4.51 (bs, 2H), 3.68-3.71 (m, 2H), 2.74-2.77 (m, 2H), 1.44 (s, 9H).

Step D

To a solution of the title compound from Step C above (4 g, 13.7 mmol) in THF (40 mL) was added sodium hydride (9.9 g, 41.23 mmol), followed by p-toluenesulfonyl chloride (2.88 g, 15.1 mmol). The reaction mixture was stirred for 30 min. The mixture was dissolved in EtOAc (200 ml) and washed with water and brine and dried over Na₂SO₄. The crude product was purified on a silica gel column using a Biotage Isolera One purification system employing an EtOAc/heptane gradient (20/80=>80/20) to afford the title compound (5 g, 82%).

MS: 445 (M+H)⁺.

¹H-NMR (400 MHz, chloroform-d) δ=7.92-7.94 (m, 1H), 7.68-7.70 (m, 1H), 7.25-7.29 (m, 4H), 7.00-7.04 (m, 1H), 4.50 (bs, 2H), 3.76 (bs, 2H), 3.12 (bs, 2H), 2.38 (s, 3H), 1.51 (s, 9H).

Step E

To a solution of the title compound from Step D above (3 g, 6.76 mmol) in dichloromethane (30 mL) was added 2N HCl (15 mL) in 1,4-dioxane. The reaction mixture was stirred for 12 h. After the completion of the reaction, the reaction mixture was evaporated to remove the solvent and washed with diethyl ether to afford the title compound as an off white solid (2 g, 78%).

MS: 345 (M+H)⁺.

¹H-NMR (400 MHz, DMSO-d₆) δ=9.50 (bs, 2H), 7.80-7.87 (m, 2H), 7.78 (d, 1H), 7.57-7.61 (m, 1H), 7.40 (d, 2H), 7.18-7.23 (m, 1H), 4.27 (bs, 2H), 3.56 (bs, 2H), 3.47 (bs, 2H), 2.34 (s, 3H).

### Preparative Example 4

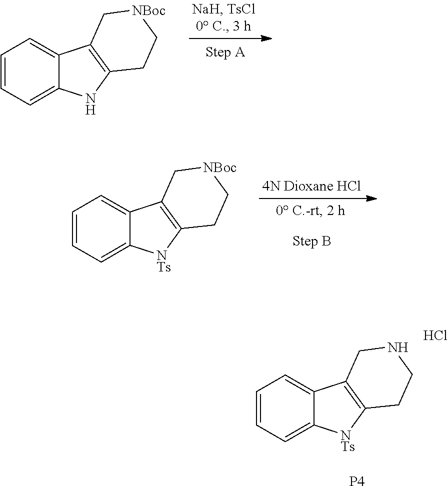

Step A

To a stirred suspension of NaH (7.65 g, 60% mineral oil, 0.191 mol) in dry THF (100 mL) at 0° C., a solution of commercially available tert-butyl 1,3,4,5-tetrahydro-2H-pyrido[4,3-b]indole-2-carboxylate (18.0 g, 0.0637 mol) in dry THF (100 mL) was added slowly and stirred at the same temperature for 60 min. Then a solution of p-toluenesulfonyl chloride (15.8 g, 0.0828 mol) in dry THF (10 mL) was added dropwise at 0° C., and the reaction mixture was allowed to stir at 0° C. for 3 h. After completion of the reaction (monitored by TLC), the reaction mixture was cooled to 0° C. and quenched with ice water (40 mL), followed by extraction using ethyl acetate (200 mL×3). The combined organic extracts were washed with water (100 mL), brine (100 mL) and dried over Na₂SO₄. The organic layer was filtered and evaporated under reduced pressure to afford the crude product which was triturated with hexane (100 mL). The solid thus obtained was filtered, washed with hexane (200 mL×2) and dried to afford the title compound as a pale brown solid (26 g, 95%).

¹H-NMR (400 MHz, DMSO-d₆) δ=8.04 (d, 1H), 7.77 (d, 2H), 7.48 (d, 1H), 7.32-7.34 (m, 3H), 7.24-7.26 (m, 1H), 4.46 (s, 2H), 3.68-3.70 (m, 2H), 3.09 (s, 2H), 2.31 (s, 3H), 1.43 (s, 3H).

MS: 327.1 (M-Boc)⁺.

Step B

To a solution of the title compound from Step A above, (26 g, 0.0603 mol) in DCM (200 mL) at 0° C., HCl in dioxane (4M, 50 mL) was added and stirred at 25° C. for 12 h. After the completion of the reaction (monitored by LCMS), the reaction mixture was evaporated under reduced pressure to yield the residue. The residue was washed with diethyl ether (100 mL×3) and dried to afford the title compound (HCl salt) (22.0 g, 99.5%) as a pale yellow solid.

¹H-NMR (400 MHz, DMSO-d₆): δ=9.92 (bs, 2H), 7.96 (d, 1H), 7.78 (d, 2H), 7.50 (d, 1H), 7.24-7.26 (m, 4H), 4.24 (s, 2H), 3.50-3.52 (m, 3H), 3.43-3.44 (m, 2H), 3.33-3.36 (m, 2H), 2.28 (s, 3H).

MS: 327.2 (M+H)⁺.

### Preparative Example 5

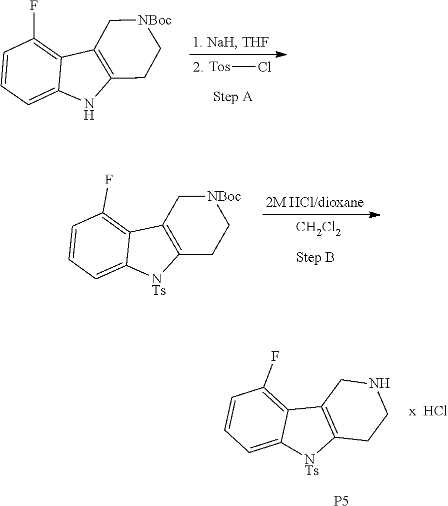

Step A

To a solution of the first eluting title compound from Preparative Example 2 of Step C (0.2 mg, 0.67 mmol) in THF (5 mL) was added sodium hydride (0.048 g, 2.137 mmol), followed by p-toluenesulfonyl chloride (0.144 g, 0.76 mmol). The reaction mixture was stirred for 30 minutes. The mixture was dissolved in EtOAc (20 ml) and washed with water and brine and dried over Na₂SO₄. The crude product was purified on a silica gel column using a Biotage Isolera One purification system employing an EtOAc/heptane gradient (20/80=>80/20) to afford the title compound (0.155 g, 50%).

MS: 445 (M+H)⁺.

¹H-NMR (400 MHz, DMSO-d₆) δ=7.80-7.82 (m, 3H), 7.31-7.32 (m, 3H), 7.07-7.09 (m, 1H), 4.56 (s, 2H), 3.68-3.69 (m, 2H), 3.09 (bs, 2H), 2.33 (s, 3H), 1.43 (s, 9H).

Step B

To a solution of the title compound from Step A above (0.15 g, 0.337 mmol) in dichloromethane was added 2N HCl (5 mL) in 1,4-dioxane. The reaction mixture was stirred for 12 h. After the completion of the reaction, the reaction mixture was evaporated to remove the solvent and washed with diethyl ether to afford the title compound as an off white solid (0.1 g, 71%).

MS: 345 (M+H)⁺.

¹H-NMR (400 MHz, DMSO-d₆) δ=9.49 (bs, 2H), 7.85-7.87 (m, 3H), 7.35-7.36 (m, 3H), 7.12-7.14 (m, 1H), 4.39 (s, 2H), 3.48 (bs, 2H), 3.17 (bs, 2H), 2.35 (s, 3H).

### Preparative Example 6

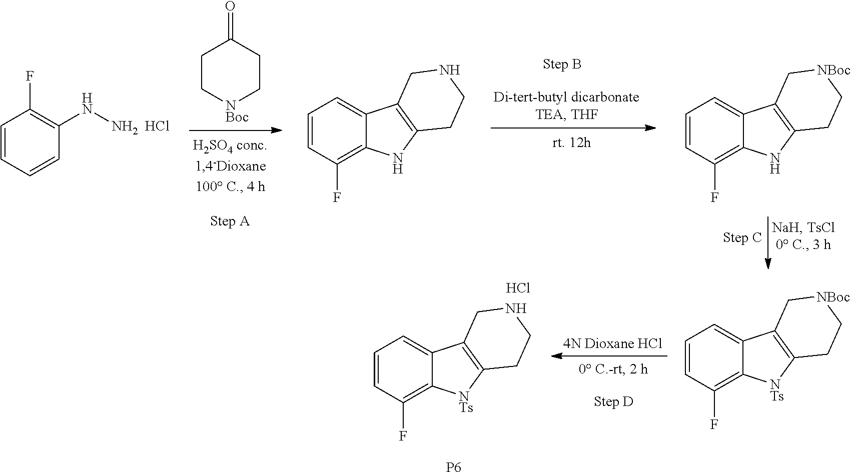

Step A

To a solution of commercially available (2-fluorophenyl)hydrazine hydrochloride (2 g, 12.34 mmol) and tert-butyl 4-oxopiperidine-1-carboxylate (2.45 g, 12.34 mmol) in dioxane (20 mL) was added concentrated H₂SO₄ (2 mL), 0° C. Then the reaction mixture was heated at 100° C. for 4 h. After completion of the reaction (monitored by TLC), the reaction mixture was cooled to 25° C. and then concentrated. The crude mixture was basified by 10% NaOH solution and the precipitate was filtered off. The solid was washed with water and dried under vacuum to get the title compound (1.7 g, 75%)_{.}

MS: 191.0 (M+H)⁺.

Step B

To a stirred solution of title compound from Step A above (1.7 g, crude) in THF (20 mL) was added TEA (3.76 mL, 26.82 mmol) and di-tert-butyl dicarbonate (2.34 mL, 10.73 mmol) at room temperature. The mixture was stirred for 12 h. After completion of the reaction (monitored by TLC), the solvent was removed and the crude reaction mixture was purified on a silica gel column using a Biotage Isolera One purification system employing an EtOAc/heptane gradient (10/80=>80/20) to afford the title compound as a pale yellow solid (0.7 g, 27%).

MS: 291.1 (M+H)⁺.

¹H-NMR (400 MHz, DMSO-d₆) δ=11.38 (bs, 1H), 7.22 (d, 1H), 6.96-6.85 (m, 2H), 4.53 (s, 2H), 3.70-3.71 (m, 2H), 2.78 (bs, 2H), 1.44 (s, 9H).

Step C To suspension of NaH (0.144 g, 3.61 mmol) in THF (15 mL) was added the title compound from Step B above (0.7 g, 2.41 mmol) (dissolved in THF) dropwise at 0° C. Then the mixture was stirred at room temperature for 1 h. After that tosyl chloride (0.549 g, 2.89 mmol) (dissolved in THF) was added at 0° C. and then the mixture was stirred at room temperature for 2 h. After completion of the reaction as evidenced by TLC, the reaction mixture was quenched with ice water, followed by extraction using ethyl acetate. The organic layer was concentrated and the crude reaction mixture was purified on a silica gel column using a Biotage Isolera One purification system employing an EtOAc/hexane gradient (10/80=>80/20) to afford the title compound (0.9 g, 84%).

MS: 345.1 (M-Boc).

¹H-NMR (400 MHz, DMSO-d₆) δ=7.79 (d, 2H), 7.41 (d, 2H), 7.34 (d, 2H), 7.22-7.23 (m, 1H), 7.07-7.09 (m, 1H), 4.50 (s, 2H), 3.70-3.72 (m, 2H), 3.17 (bs, 2H), 2.36 (s, 3H), 1.40 (s, 9H).

Step D

To a solution of the title compound from Step C above (0.9 g, 2.02 mmol) in dichloromethane (10 mL) was added 2N HCl (5 mL) in dioxane. The reaction mixture was stirred at room temperature for 2 h. After the completion of the reaction, the reaction mixture was evaporated to remove the solvent and the residue was washed with diethyl ether to afford the title compound as a pale brown solid (0.45 g, 65%). MS: 345.1 (M+H)⁺.

### Preparative Example 7

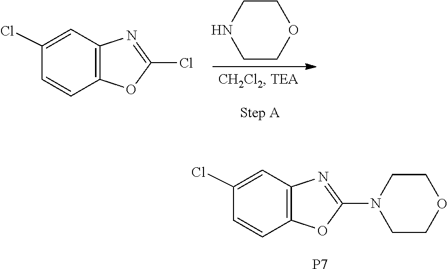

Step A

To a stirred solution of 2,5-dichlorobenzo[d]oxazole, (150 g, 0.8 mol, 1.0 equiv) in DCM (1.5 L) at 0° C., triethylamine (336 mL, 2.39 mol, 3.0 equiv) and morpholine (83.4 g, 0.95 mol, 1.2 equiv) were added at 0° C., then the reaction mixture was stirred at 25° C. for 12 h. After completion of the reaction (monitored by TLC), the reaction mixture was quenched with water (250 mL) and extracted with dichloromethane (500 mL×3). The combined organic extracts were dried over Na₂SO₄, filtered and evaporated under reduced pressure to afford the crude product. It was triturated with methyl tert-butyl ether (MTBE, 300 mL) and the solid thus obtained was filtered, washed with MTBE (100 mL×2) and dried to afford the title compound as an off-white solid (175 g, 92%).

¹H-NMR (400 MHz, DMSO-d₆) δ=7.44 (d, 1H), 7.36 (d, 1H), 7.06 (dd, 1H), 3.71-3.74 (m, 4H), 3.59-3.61 (m, 4H).

MS: 239.0 (M+H)⁺.

### Preparative Example 8

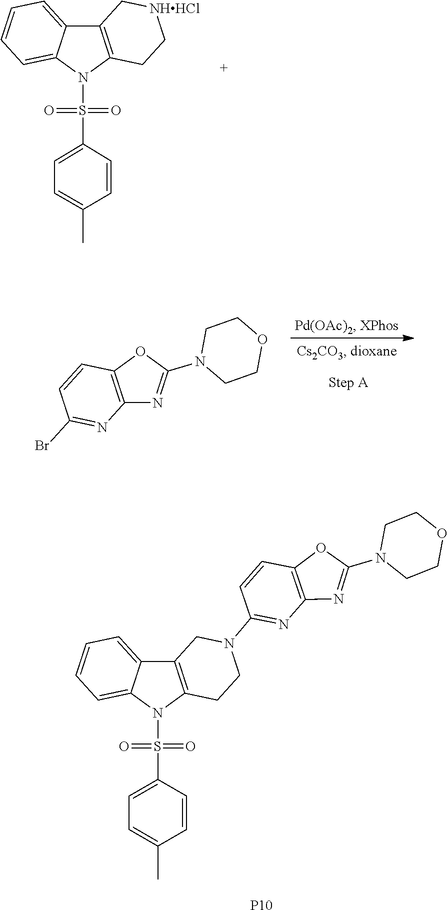

Step A

Palladium (II) acetate (0.613 g, 0.00273 mol) and 2-dicyclohexylphosphino-2′,4′,6′-triisopropylbiphenyl (XPhos) (3.90 g, 0.00819 mol) were placed in a reaction vial and degassed 1,4-dioxane (200 mL) was added. The resulting solution was degassed briefly. The suspension was heated at 100° C. (on a pre-heated heating block) for less than 1 minute until the color of the solution turned from orange to dark pink. Then, the vial was removed from the heating block and the title compound from Preparative Example 4 (10.0 g, 0.0273 mol) and the title compound from Preparative Example 7 (5.41 g, 0.0227 mol) and caesium carbonate (26.7 g, 0.0819 mol) were added. The reaction vial was filled with argon before closing it. The reaction mixture was heated at 100° C. for 12 h. After the completion of the reaction (monitored by TLC), the reaction mixture was filtered through celite washed with ethyl acetate (200 mL×3) and the filtrate was concentrated under reduced pressure to yield the crude product which was purified by silica gel (230-400 mesh) column chromatography using hexane: EtOAc (35:65) to afford the title compound as a pale yellow solid (9.0 g, 61.6%).

¹H-NMR (400 MHz, DMSO-d₆): δ=8.03 (d, 1H), 7.71 (d, 2H), 7.57 (d, 1H), 7.26-7.28 (m, 5H), 7.05-7.06 (m, 1H), 6.79-6.80 (m, 1H), 4.27 (s, 2H), 3.71-3.72 (m, 4H), 3.55-3.57 (m, 6H), 3.18 (bs, 2H), 2.30 (s, 3H).

MS: 529.2 (M+H)⁺.

### Preparative Example 9

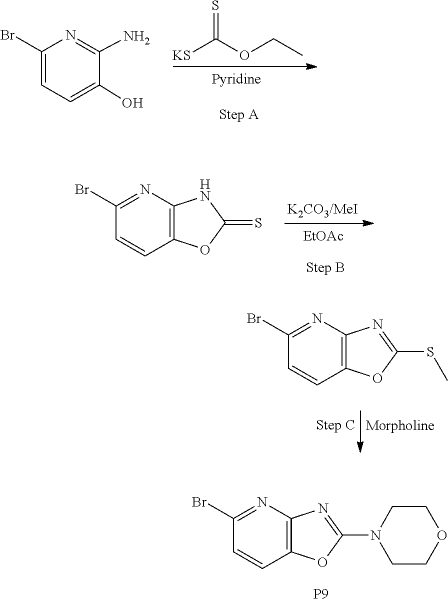

Step A:

To a stirred solution of 2-amino-6-bromopyridin-3-ol (2.5 g, 13.3 mmol) in pyridine (25 mL) was added potassium ethyl xanthogenate (6.39 g, 39.9 mmol) and heated to 100° C. for 12 h. After completion of the reaction (monitored by TLC), the reaction mixture was acidified with 12N HCl, the solid precipated out was filtered and washed with water to give the desired product (2.4 g, crude) as gummy brown solid. LCMS=231 (M−H)⁺. The crude product was taken as such for next step.

Step B:

To a stirred solution of 5-bromooxazolo[4,5-b]pyridine-2(3H)-thione (2.4 g, 10.4 mmol) in ethyl acetate (20 mL) at 25° C., was added potassium carbonate (2.01 g, 14.6 mmol) and stirred at RT for 10 mins. After 10 mins, the reaction mixture was cooled to 0° C., and methyl iodide (0.94 mL, 14.6 mmol) was slowly added. The reaction mixture was stirred at RT for 12 hours. After completion of the reaction (monitored by TLC) the reaction mixture was poured into water (50 mL) and extracted with ethyl acetate (2×50 mL). The combined organic layer was washed with saturated brine solution (1×50 mL), dried over sodium sulfate and concentrated under vacuo to afford the desired product (2.3 g, 92%) as brown solid. LCMS=245 (M+H)⁺. 1H NMR (300 MHz, DMSO-d₆): δ 8.07 (d, J=8.40 Hz, 1H), 7.55 (d, J=8.40 Hz, 1H), 2.80 (s, 3H).

Step C:

A solution of 5-bromo-2-(methylthio)oxazolo[4,5-b]pyridine (2.3 g, 9.4 mmol) in morpholine (25 mL) was heated at 80° C. for 12 hours. After completion of the reaction (monitored by TLC), the reaction mixture was diluted with ethyl acetate (50 mL). The organic layer was washed with water (2×20 mL), dried over sodium sulfate and concentrated under vacuo to afford the desired product (2 g, 76%) as a brown solid.

LCMS=284.0 (M+H)⁺.

¹H NMR (400 MHz, DMSO-d₆) δ 7.74 (d, J=8.40 Hz, 1H), 7.21 (d, J=8.00 Hz, 1H), 3.72-3.73 (m, 4H), 3.65-3.66 (m, 4H),

### Preparative Example 10

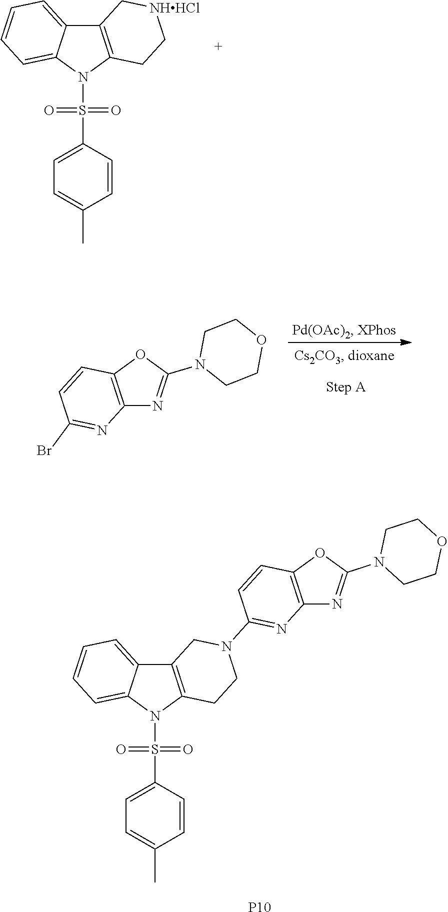

Pd(OAc)₂ (20.7 mg, 0.0917 mmol) and 2-Dicyclohexylphosphino-2′,4′,6′-triisopropylbiphenyl (XPhos; 131 mg, 0.2752 mmol) were added to a reaction vial and then deglazed dioxane (6 ml) was added. The vial was filled with Argon gas and sealed. The suspension was heated to 100° C. for 1 minute then the title compound from Preparative Example 4 (300 mg, 0.9174 mmol), the title compound from Preparative Example 9 (286 mg, 1.0091 mmol) and Cs₂CO₃ (904 mg, 2.7523 mmol) were added, and the solution was heated at 100° C. for 12 hours. The reaction mixture was diluted with ethyl acetate (30 mL) and water (30 mL). The organic phase was separated, and the aqueous phase was extracted with ethyl acetate two more times. The combined organic phase was dried over Na₂SO₄, filtered and the solvents were evaporated under reduced pressure. The crude product was purified on HP-Sil column (Biotage), by employing a EtOAc/Hexane gradient (0-45%) to afford the Tosyl protected compound (80 mg, 16.49%). LCMS=530.0 (M+H)⁺. ¹H-NMR (400 MHz, DMSO-d₆) δ=8.04 (d, 1H), 7.73-7.74 (m, 2H), 7.64 (d, 1H), 7.57 (d, 1H), 7.28-7.29 (m, 4H), 6.60 (d, 1H), 4.55 (s, 2H), 3.93-3.94 (m, 2H), 3.71-3.73 (m, 4H), 3.59-3.60 (m, 4H), 3.19 (bs, 2H), 2.33 (s, 3H),

### Example 1

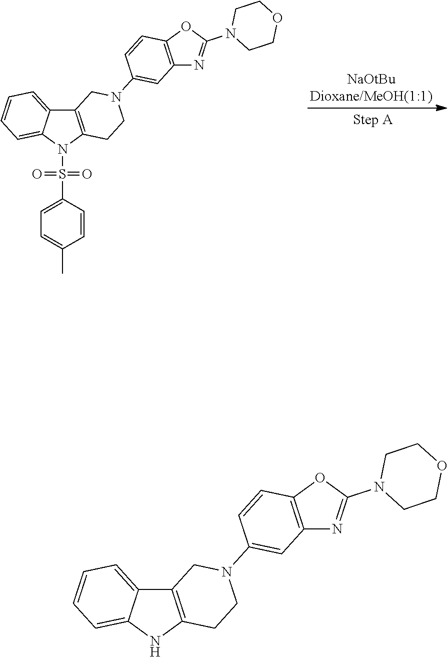 [SMILES: Cc1ccc(S(=O)(=O)n2c3c(c4ccccc42)CN(c2ccc4oc(N5CCOCC5)nc4c2)CC3)cc1 ; c1ccc2c3c([nH]c2c1)CCN(c1ccc2oc(N4CCOCC4)nc2c1)C3]

Step A

To a stirred solution of the title compound from Preparative Example 8 (9 g, 0.0168 mol) in 1,4-dioxane:MeOH (1:1, 300 mL), was added sodium tert-butoxide (8.07 g, 0.0840 mol). The reaction mixture was heated to 70° C. for 12 h under nitrogen. The reaction was monitored by LCMS. The crude LCMS shows 95% of product mass and 5% of starting material. Then another 4 g of sodium tert-butoxide was added and heated to 70° C. for 6 h. After completion, the reaction mixture was concentrated under vacuum and the crude product was diluted with dichloromethane (300 mL) and water (300 mL). The organic phase was separated and the aqueous phase was extracted with dichloromethane one more time. The combined organic phase was dried over Na₂SO₄, filtered and the solvents were evaporated under reduced pressure. The crude product was added to methanol (50 mL), stirred for 30 min and then filtered, washed with diethyl ether (50 mL) and dried under vacuum for 6 h to afford the title compound (6.0 g, 94.1%) as a pale yellow solid.

The compound was dissolved in dry THF (500 mL) and passed through celite, washed with THF (repetition of the process 3 times) and evaporated under reduced pressure to afford the product which was further recrystallized from methanol to afford the title compound as a pale yellow solid (95% yield).

¹H-NMR (400 MHz, DMSO-d₆): δ=10.84 (bs, 1H), 7.46 (d, 1H), 7.25-7.28 (m, 2H), 7.01-7.03 (m, 3H), 6.77-6.78 (m, 1H), 4.34 (s, 2H), 3.70-3.71 (m, 4H), 3.63 (t, 2H), 3.54-3.56 (m, 4H), 2.88 (t, 2H).

MS: 375.1 (M+H)⁺.

### Comparative Examples 1 to 3, General Procedure

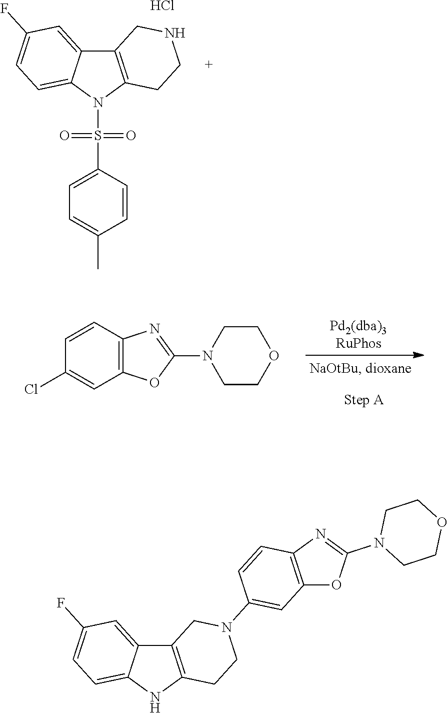 [SMILES: Cl ; Cc1ccc(S(=O)(=O)n2c3c(c4cc(F)ccc42)CNCC3)cc1 ; Clc1ccc2nc(N3CCOCC3)oc2c1 ; Fc1ccc2[nH]c3c(c2c1)CN(c1ccc2nc(N4CCOCC4)oc2c1)CC3]

Step A To a stirred solution of the title compound of Preparative Example 1 (0.150 g, 1 eq) in dry 1,4-dioxane (5 mL) was added the title compound of Preparative Example 7 (1 eq), sodium tert-butoxide (3 eq) and the mixture was degassed for 10 minutes under N₂ atmosphere. To this reaction mixture was added Pd2(dba)₃ (0.05 eq) and Ru-Phos (0.1 eq) and the mixture was heated to 100° C. until the completion of the reaction. After the completion of the reaction, the reaction mixture was filtered through a celite bed, and washed with EtOAc. The filtrate was concentrated and the crude product was purified by column chromatography or preparative HPLC to afford the title compound as indicated in the following table.

### Comparative Examples 1 to 3

Following the palladium coupling procedures as described in the general procedure above, the following compounds were prepared.

TABLE 1

| | Tricyclic | Bromo or | | 1. Yield |
|---|---|---|---|---|
| Comparative | amino | chloro | | 2. ¹H-NMR |
| Example | derivative | derivative | Product | 3. MH⁺ (ESI) |
| 1 | 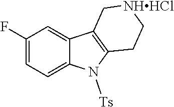 | 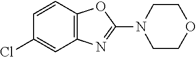 [SMILES: Clc1ccc2oc(N3CCOCC3)nc2c1] | 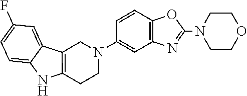 [SMILES: Fc1ccc2[nH]c3c(c2c1)CN(c1ccc2oc(N4CCOCC4)nc2c1)CC3] | 1. 24% 2. ¹H-NMR (400 MHz, DMSO- d₆) δ = 7.29-7.23 (m, 3H), 7.04 (d, 1H), 6.88-6.83 (m, 1H), 6.80-6.77 (m, |
| | | | | 1H), 4.31 (s, 2H), |
| | | | | 3.73-3.71 (m, 4H), |
| | | | | 3.64-3.61 (m, |
| | | | | 2H), 3.56 (t, 4H), |
| | | | | 2.88-2.87 (m, 2H). |
| | | | | 3. 393.4 |
| 2 | 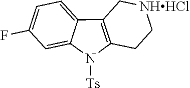 | 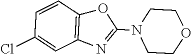 [SMILES: Clc1ccc2oc(N3CCOCC3)nc2c1] | 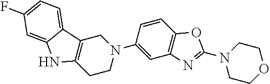 [SMILES: Fc1ccc2c3c([nH]c2c1)CCN(c1ccc2oc(N4CCOCC4)nc2c1)C3] | 1. 15% 2. ¹H-NMR (400 MHz, DMSO-d₆) δ = 10.94 (bs, 1H), 7,4-7.45 (m, 1H), |
| | | | | 7.27 (d, 1H), |
| | | | | 7.04-7.08 (m, 2H), |
| | | | | 6.77-6.85 (m, 2H), |
| | | | | 4.33 (bs, 2H), 3.72 |
| | | | | (bs, 4H), 3.56-3.61 |
| | | | | (m, 6H), 2.86 |
| | | | | (bs, 2H). |
| | | | | 3. 393.2 |
| 3 | 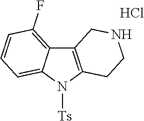 [SMILES: Cc1ccc(S(=O)(=O)n2c3c(c4c(F)cccc42)CNCC3)cc1 ; Cl] | 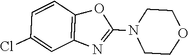 [SMILES: Clc1ccc2oc(N3CCOCC3)nc2c1] | 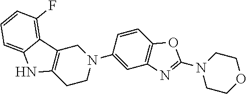 [SMILES: Fc1cccc2[nH]c3c(c12)CN(c1ccc2oc(N4CCOCC4)nc2c1)CC3] | 1. 8% 2. ¹H-NMR (400 MHz, DMSO-d₆) δ = 11.17 (bs, 1H), 7.27 (d, 1H), 7.13 (d, 1H), 6.98-7.01 (m, |
| | | | | 2H), 6.70-6.78 |
| | | | | (m, 2H), 4.44 |
| | | | | (bs, 2H), 3.70- |
| | | | | 3.73 (m, 4H), |
| | | | | 3.61-3.63 (m, |
| | | | | 2H), 3.56-3.57 |
| | | | | (m, 4H), 2.86- |
| | | | | 2.88 (m, 2H). |
| | | | | 3. 393.0 |

### Comparative Example 4

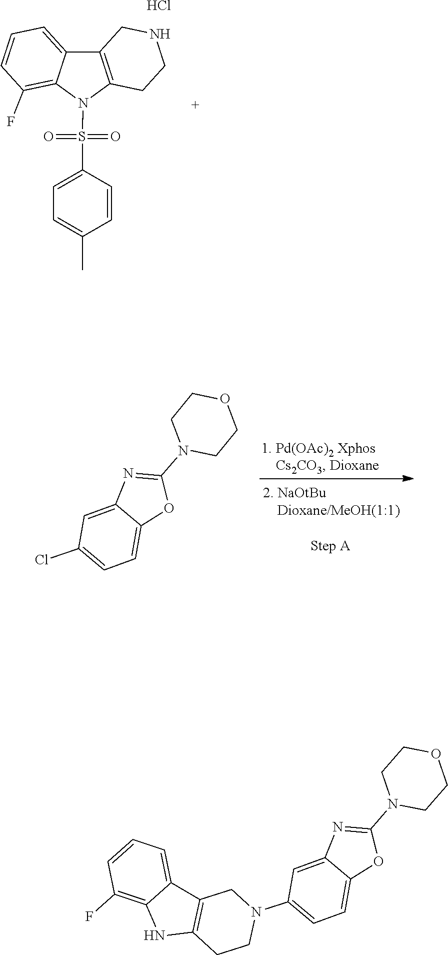 [SMILES: Cl ; Cc1ccc(S(=O)(=O)n2c3c(c4cccc(F)c42)CNCC3)cc1 ; Clc1ccc2oc(N3CCOCC3)nc2c1 ; Fc1cccc2c3c([nH]c12)CCN(c1ccc2oc(N4CCOCC4)nc2c1)C3]

Step A

To a stirred solution of the title compound from Preparative Example 6 (0.15 g, 0.43 mmol) in dry 1,4-dioxane (5 mL), was added the title compound from Preparative Example 7 (0.17 g, 0.43 mmol) and Cs₂CO₃ (0.420 g, 1.29 mmol). The reaction mixture was degassed for 10 min under N₂ atmosphere. Then Pd(OAc)₂ (0.009 g, 0.043 mmol) and 2-dicyclohexylphosphino-2′,4′,6′-triisopropylbiphenyl (0.062 g; 0.129 mmol) were added and the reaction mixture was heated to 100° C. until the completion of the reaction. After the completion of the reaction (monitored by LCMS), the reaction mixture was filtered through celite and washed with ethyl acetate. The filtrate was concentrated under reduced pressure to yield the crude product. The crude material was purified by flash column chromatography or preparative HPLC to afford the tosyl protected compound. To a solution of tosyl compound (1.0 eq) in 1,4-dioxane:MeOH (1:1, 10 vol) was added NaOtBu (3 eq). The reaction mixture was heated to 70° C. for 6 hours. The reaction mixture was concentrated under vacuum and the crude product was column purified to afford the desired product. The crude material was purified by flash column chromatography or preparative HPLC to afford the title compound (0.049 g, 49%).

¹H-NMR (400 MHz, DMSO-d₆): δ=11.29 (s, 1H), 7.25-7.27 (m, 2H), 7.04 (d, 1H), 6.77-6.94 (m, 3H), 4.34 (s, 2H), 3.70-3.71 (m, 4H), 3.61-3.62 (m, 2H), 3.54-3.55 (m, 4H), 2.87-2.89 (m, 2H).

MS: 393.2 (M+H)⁺.

### Example 5

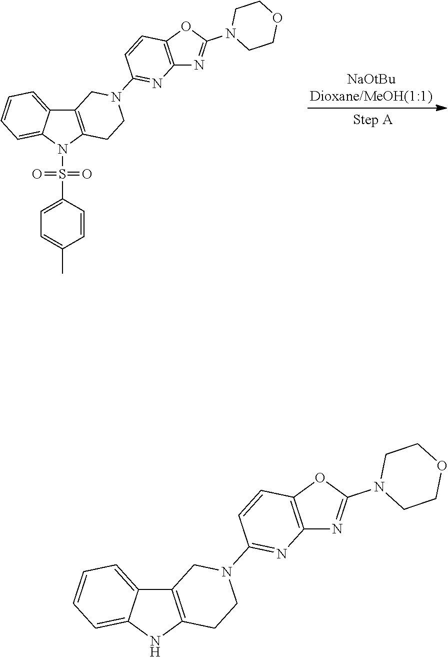 [SMILES: Cc1ccc(S(=O)(=O)n2c3c(c4ccccc42)CN(c2ccc4oc(N5CCOCC5)nc4n2)CC3)cc1 ; c1ccc2c3c([nH]c2c1)CCN(c1ccc2oc(N4CCOCC4)nc2n1)C3]

To a stirred solution of the title compound from Preparative Example 10 (80 mg, 0.1512 mmol)) in 1,4-dioxane:MeOH (1:1, 5 mL), was added sodium tert-butoxide (43.62 mg, 0.4536 mmol). The reaction mixture was heated to 70° C. for 12 h under nitrogen. After completion, the reaction mixture was concentrated under reduced pressure and the crude product purified on HP-Sil column (Biotage), by employing a EtOAc/Hexane gradient (0-100%) to afford the title compound (50 mg, 89%) as yellow solid. LCMS=376.1 (M+H)⁺.

¹H-NMR (400 MHz, DMSO-d₆) δ=10.86 (bs, 1H), 7.60 (d, 1H), 7.47 (d, 1H), 7.29 (d, 1H), 6.98-6.99 (m, 2H), 6.56 (d, 1H), 4.63 (s, 2H), 3.97 (t, 2H), 3.71-3.72 (m, 4H), 3.59-3.60 (m, 4H), 2.88-2.90 (m, 2H).

Biological Assay Description

Tau K18 Disaggregation Assay by ThT

The Tau K18 fragment, encompassing amino acids 244 to 372 of the longest isoform (2N4R) of human Tau441, was expressed in bacteria and purified or bought from SignalChem. For the K18 disaggregation assay by ThT, 35 μM of recombinant K18 in PBS were aggregated for 24 hours at 37° C. in presence of 50 μM of heparin (Sigma-Aldrich) and 10 mM of DTT (Sigma-Aldrich) under shaking at 750 RPM. Compounds were dissolved in anhydrous dimethyl sulfoxide (DMSO, Sigma-Aldrich) to reach a concentration of 10 mM. K18 aggregates and serial dilutions of compounds were mixed together in PBS (volume 50 μL) to a final concentration of 2 μM of K18 aggregates and from 160 to 0.04 μM of compounds. The mixture was incubated for 30 minutes at room temperature (RT), then 40 μL of this mixture were transferred into a black 384-well plate assay (Perkin-Elmer) and mixed with 10 μL of 100 μM ThT in 250 mM glycine (both from Sigma-Aldrich) in PBS. Fluorescence (relative fluorescence units; RFU) was measured in monoplicate or duplicate on a Tecan reader (excitation: 440 nm; emission: 485 nm). Percentage of K18 disaggregation was then calculated and half maximal effective concentration (EC₅₀) was determined using GraphPad Prism version 5 (GraphPad Software) assuming a one-binding site fitting model. As shown in Table 2, all compounds measured showed high potency of disaggregation of Tau K18.

The following example compounds were measured:

TABLE 2

| | Tau K18 |
|---|---|
| Example/Comparative | disaggregation EC₅₀ |
| Examples | (μM) |
| Example 1 | +++ |
| Comparative Example | +++ |
| 1 | |
| Comparative Example | +++ |
| 2 | |
| Comparative Example | +++ |
| 3 | |
| Comparative Example | +++ |
| 4 | |
| Example 5 | +++ |

Legend:
+++ EC₅₀ <10 μM;
++ EC₅₀ 10 < x < 25 μM;
+ EC₅₀ 25 < x < 50 μM.

Tau disaggregation assay by DLS (dynamic light scattering)

The longest isoform of human Tau (2N4R; 441 amino acids) was expressed in bacteria. For the Tau disaggregation assay by DLS, 35 μM of recombinant full-length (fl)Tau in PBS were aggregated for 24 h at 37° C. in presence of 50 μM of heparin (Sigma-Aldrich) and 10 mM of DTT (Sigma-Aldrich) under shaking at 750 RPM. Morphomers™ were dissolved in DMSO (Sigma-Aldrich) to reach a concentration of 10 mM. flTau aggregates and compounds were mixed together in PBS (volume 80 μL) to a final concentration of 2 μM of flTau aggregates and 20 μM of Morphomers™. The mixture was incubated for 60 min at RT. Then 70 μl of solution was transferred into a 40 μl cuvette (Malvern) and the size of flTau aggregates was measured with a Zetasizer Nano (Malvern) at an angle of 173° with 3 runs of 5 times 15 sec each. Number of events were taken into consideration to assess size of flTau aggregates. Percentage of disaggregation was then measured using the dynamic range between flTau monomers and flTau aggregates. As shown in Table 3, the compounds measured showed efficacy of reducing the size of full-length Tau aggregates.

The following example compound were measured:

TABLE 3

| | DLS % |
|---|---|
| | Percentage of |
| | disaggregation |
| Example | (flTau) |
| 1 | 25 |

Legend:
n.d. not determined.

CSF/Plasma Ratio Determination

Test compounds were administered to CD-1 male mice as a single oral dose of 10 or 20 mg/kg of a homogenous suspension (10 mL/kg, 1 or 2 mg/mL in 0.5% CMC (w/v) in water) by gavage using a plastic syringe fitted with a metal gavage tube. Terminal blood, brain and CSF samples were collected from three animals at several time points, typically at 2, 4, 8 and 24 h post-dose.

Blood samples were transferred into 1.5-mL plastic tubes containing K2-EDTA (0.5M, 3 uL) as an anti-coagulant and placed on wet ice, and were then processed to obtain plasma by centrifugation at approximately 5° C. (3000 g, 15 min) within 30 min after collection. An aliquot of 10 μL sample was protein precipitated with 150 μL IS, the mixture was vortex-mixed well and centrifuged at 13000 rpm for 10 min, 4° C. 30 μL supernatant was then mixed with 30 μL water, vortex-mixed well and centrifuged at 4° C. 2 μL sample was injected for LC-MS/MS analysis.

The collected CSF was transferred to polypropylene microcentrifuge tubes. CSF samples were added with an equal volume of plasma. An aliquot of 6 μL sample was protein precipitated with 90 μL IS, the mixture was vortex-mixed well and centrifuged at 13000 rpm for 10 min, 4° C. 30 μL supernatant was then mixed with 30 μL water, vortex-mixed well and centrifuged at 4° C. 2 μL sample was injected for LC-MS/MS analysis.

Test substance concentration in plasma and CSF versus time data were analyzed by non-compartmental approaches; AUC0-last in plasma and CSF were calculated with the log-linear trapezoidal rule. CSF/Plasma ratio was calculated as follows:

AUC0-last(CSF)/AUC0-last(plasma)

As shown in Table 4, the compound 1 showed the highest CSF/plasma ratio, which is the important parameter for central nervous system (CNS) availability of the compound.

TABLE 4
In-vivo Pharmacokinetics Mouse p.o. PK (20 mg/kg)

| | | CSF | |
|---|---|---|---|
| Example/Comparative | Plasma AUC0- | AUC0-last | CSF/Plasma |
| Examples | last (ng · h/mL) | (ng · h/mL) | ratio |
| 1 | 23656.8 | 327.0 | 0.014 |
| Comparative Example 1 | 17443.7 | 121.03 | 0.007 |
| Comparative Example 2 | 17757.6 | 56.70 | 0.003 |
| Comparative Example 3* | 4591.92 | 19.705 | 0.004 |
| Comparative Example 4 | 14467.2 | 66.5 | 0.005 |

*In-vivo Pharmacokinetics Mouse p.o. PK 10 (mg/kg)

1. A compound of formula (I): 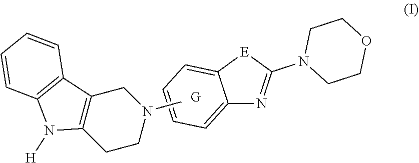
or a pharmaceutically acceptable salt thereof;
wherein
E is selected from the group consisting of O and S; and
G is selected from the group consisting of a benzene ring, a pyrimidine ring and a pyridine ring.

2. The compound according to claim 1, which is a compound of formula (Ia): 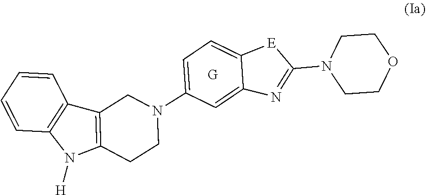
wherein E, and G are as defined in claim 1.

3. The compound according to claim 1, wherein G is a benzene ring.

4. The compound according to claim 1, wherein E is O.

5. A pharmaceutical composition comprising a compound as defined in claim 1 and optionally a pharmaceutically acceptable carrier or excipient.

6. The compound as defined in claim 1 for use as a medicament.

7. The compound as defined in claim 1 for use in the treatment, alleviation or prevention of a disorder or abnormality associated with Tau protein aggregates.

8. The compound as defined in claim 1 for use in decreasing tau aggregation.

9. A method of treating, preventing or alleviating a disorder or abnormality associated with Tau protein aggregates, the method comprising administering an effective amount of a compound as defined in claim 1 to a subject in need thereof.

10. A method of decreasing Tau aggregation, the method comprising administering an effective amount of a compound as defined in claim 1 to a subject in need thereof.

11-12. (canceled)

13. A mixture comprising a compound as defined in claim 1 and at least one further biologically active compound selected from a therapeutic agent different from the compound as defined in claim 1,
wherein the mixture further comprises at least one of a pharmaceutically acceptable carrier, a diluent and an excipient.

14. The mixture according to claim 13, wherein the further biologically active compound is a compound used in the treatment of amyloidosis.

15. The mixture according to claim 13, wherein the compound and/or the further biologically active compound is/are present in a therapeutically effective amount.

16. The mixture according to claim 13, wherein the further biologically active compound is selected from the group consisting of compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), α-secretase activators, β- and γ-secretase inhibitors, glycogen synthase kinase 3 inhibitors, O-glucanase (OGA) inhibitors, neurotransmitter, β-sheet breakers, attractants for amyloid beta clearing/depleting cellular components, inhibitors of N-terminal truncated amyloid beta including pyroglutamated amyloid beta 3-42, anti-inflammatory molecules, or cholinesterase inhibitors (ChEIs) such as tacrine, rivastigmine, donepezil, and/or galantamine, M1 agonists, other drugs including any amyloid or Tau modifying drug and nutritive supplements, an antibody, including any functionally equivalent antibody or functional parts thereof or a vaccine.

17. The mixture according to claim 16, wherein the further biologically active compound is a cholinesterase inhibitor (ChEI).

18. The mixture according to claim 16, wherein the further biologically active compound is selected from the group consisting of tacrine, rivastigmine, donepezil, galantamine, niacin and memantine.

19. The mixture according to claim 16, wherein the further biologically active compound is an antibody, particularly a monoclonal antibody, including any functionally equivalent antibody or functional parts thereof.

20. The mixture according to claim 13, wherein the compound and/or the further biologically active compound is/are present in a therapeutically effective amount.

21. The method according to claim 9 wherein the disorder is selected from Alzheimer's disease (AD), familial AD, Primary Age-Related Tauopathy (PART), Creutzfeldt-Jacob disease, dementia pugilistica, Down's Syndrome, Gerstmann-Sträussler-Scheinker disease (GSS), inclusion-body myositis, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17 (FTDP-17), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease, preferably Alzheimer's disease (AD), corticobasal degeneration (CBD), Pick's disease (PiD), and progressive supranuclear palsy (PSP).

22. (canceled)
